(19) United States
(12) Patent Application Publication
(10) Pub. No.: US 2023/0149309 A1
(43) Pub. Date: May 18, 2023

(54) COMPOSITION COMPRISING ANTIMICROBIAL AGENT AND ITS USES

(71) Applicant: SHARED VISION TECHNOLOGY LTD., Trim (IE)

(72) Inventors: Martin TIGHE, Trim (IE); Ivan COULTER, Mount Merrion (IE)

(73) Assignee: SHARED VISION TECHNOLOGY LTD., Trim (IE)

(21) Appl. No.: 17/915,021

(22) PCT Filed: Mar. 27, 2021
(86) PCT No.: PCT/EP2021/058056

§ 371 Date: Sep. 27, 2022

(30) Foreign Application Priority Data
Mar. 27, 2020 (GB) 2004536.5

(51) Int. Cl. A61K 9/107; A61K 31/23; A61P 31/16; A61P 11/00; A61P 1/12; A61P 31/14; A23L 33/12; A23K 20/158; A23K 50/30

(52) U.S. Cl. CPC A61K 9/1075; A61K 31/23; A61P 31/16; A61P 11/00; A61P 1/12; A61P 31/14; A23L 33/12; A23K 20/158; A23K 50/30

(57) ABSTRACT

A composition comprising 2,3 dihydroxypropyl dodecanoate, and an emulsifier results in greater dissolution and biodistribution of 2,3 dihydroxypropyl dodecanoate to enhance antimicrobial and other activities.

### FIELD OF THE INVENTION

The invention relates to a composition comprising the antimicrobial agent 2,3 dihydroxypropyl dodecanoate and its uses.

### BACKGROUND TO THE INVENTION

The active agent 2,3 dihydroxypropyl dodecanoate, also known as monolaurin, alpha monolaurin, glycerol monolaurate or 1-lauroyl-glycerol, is a monoglyceride and is known to have anti-microbial properties including anti-viral, anti-bacterial, anti-fungal and anti-protozoan properties in vitro. Due to normal digestive processes in animals, it is not optimised for oral bioavailability in its complete “active” form. In particular, the assumed normal action of lipases, such as pancreatic lipases, in the gastrointestinal tract will cleave the esterified fatty acid from the glycerol of 2,3 dihydroxypropyl dodecanoate. Cleavage of 2,3 dihydroxypropyl dodecanoate by lipases yields dodecanoic acid (lauric acid) and glycerol, both of which are readily absorbed by animals. However, dodecanoic acid maintains a fraction of certain anti-viral and general anti-microbial properties associated with 2,3 dihydroxypropyl dodecanoate.

### SUMMARY OF THE INVENTION

In one aspect the present invention provides a composition comprising a) 2,3 dihydroxypropyl dodecanoate, and b) an emulsifier. Beneficially the emulsifier of the composition enhances bioavailability of the 2,3 dihydroxypropyl dodecanoate. Beneficially the emulsifier of the composition enhances the activity of the 2,3 dihydroxypropyl dodecanoate. The emulsifier of the composition may inhibit or compete with the action of lipase on 2,3 dihydroxypropyl dodecanoate in the gastrointestinal tract preventing lipase from cleaving 2,3 dihydroxypropyl dodecanoate to dodecanoic acid and glycerol. The emulsifier of the composition may allow for rapid dissolution and uptake of 2,3 dihydroxypropyl dodecanoate in the gastrointestinal tract. Rapid dissolution within the gastric and/or intestinal environment may improve biodistribution and uptake of 2,3 dihydroxypropyl dodecanoate. The emulsifier of the composition may increase the amount of 2,3 dihydroxypropyl dodecanoate absorbed via the lymphatic system. Absorption through the lymphatic system may allow the 2,3 dihydroxypropyl dodecanoate to avoid the liver where it may be broken down to metabolites and energy.

The composition of the invention may comprise of from about 2 wt. % to about 95 wt. % 2,3 dihydroxypropyl dodecanoate based on the total weight of the composition, for example the composition may comprise of from about 5 wt. % to about 60 wt. % 2,3 dihydroxypropyl dodecanoate based on the total weight of the composition, for example from about 10 wt. % to about 20 wt. % 2,3 dihydroxypropyl dodecanoate based on the total weight of the composition.

The composition of the invention may comprise of from about 10 wt % to about 95 wt. % emulsifier based on the total weight of the composition, for example from about 40 wt. % to about 92 wt. % emulsifier based on the total weight of the composition, for example from about 80 wt. % to about 90 wt. % emulsifier based on the total weight of the composition.

The composition of the invention may comprise an emulsifier which is selected from the group of Acacia (gum arabic), Acetylated monoglycerides, Aluminum salts of fatty acids, Arabinogalactan, Bacterial Catalase, Bakers Yeast Glycan, BHA (butylated hydroxyanisole), BHT (butylated hydroxytoluene), Calcium carbonate, Calcium salts of fatty acids, Carob bean gum (locust bean gum), Curdlan, Diacetyl tartaric acid esters of mono- and diglycerides of edible fats or oils, or edible fat-forming fatty acids, Dioctyl sodium sulfosuccinate, Disodium phosphate, Sodium phosphate mono-, di-, & tri-), Enriched Farina; 136.3, Bakery Products, Ethoxylated mono- and diglycerides, Eucheuma cottonii extract, Eucheuma spinosum extract, Fatty acids, salts of (aluminum, calcium, magnesium, potassium, and sodium), Food starch esterified with n-octenyl succinic anhydride treated with beta-amylase, Furcelleran, Furcelleran, salts of ammonium, calcium, potassium, or sodium, Ghatti gum, Gigartina extracts, Glyceryl-lacto esters of fatty acids, Gum ghatti, Hexitol oleate, Hydroxylated lecithin, Hydroxypropyl cellulose, Hydroxypropyl methylcellulose, Lactylated fatty acid esters of glycerol and propylene glycol, Lactylic esters of fatty acids, Lecithin, hydroxylated lecithin, Methyl ethyl cellulose, Mono- & diglycerides of edible fats or oils, or edible fat forming acids, Monoisopropyl citrate, Monosodium phosphate derivatives of mono- & diglycerides of edible fats or oils, or edible fat-forming fatty acids, Myrj 45 (polyoxyethylene 8-stearate), Ox bile extract, Pectins (including pectin modified), Polyethylene glycol (400) dioleate, Polyglycerol esters of fatty acids, Polyoxyethylene glycol (400) mono- & di-oleates, Polysorbate 60 (Polyoxyethylene (20) sorbitan monostearate), Polysorbate 65 (Polyoxyethylene (20) sorbitan tristearate), Polysorbate 80 (Polyoxyethylene (20) sorbitan monooleate), Potassium salts of fatty acids, Propylene glycol alginate (Propylene glycol ester of alginic acid), Propylene glycol mono- & di- esters of fats & fatty acids, Rapeseed oil, fully hydrogenated, superglycerinated, Sodium acid pyrophosphate, Sodium aluminum phosphate, Sodium hypophosphite, Sodium lauryl sulfate, Sodium metaphosphate, Sodium methyl sulfate, Sodium pectinate, Sodium salts of fatty acids, Sodium stearoyl lactylate, Sodium sulfo-acetate derivatives (mono- & diglycerides), Sorbitan monoleate, Sorbitan monostearate, Succinylated monoglycerides, Succistearin (stearoyl propylene glycol hydrogen succinate), Sucrose acetate isobutyrate (SAIB), Sucrose fatty acid esters, Sucrose oligoesters, Sulfated butyl oleate, Trisodium phosphate, and Xanthan gum or a combination thereof. Preferably the emulsifier is chosen from the group of Fatty acids, Glyceryl-lacto esters of fatty acids, salts of fatty acids, mono glycerides of fatty acids, Lactylated fatty acid esters of glycerol and propylene glycol, Lactylic esters of fatty acids, sucrose oligoesters, sorbitol, polysorbitan, Lecithin, hydroxylated Lecithin, and Sodium Lauryl Sulfate, or combinations thereof.

The emulsifiers may comprise polysorbates, for example polysorbate 80. The emulsifiers may comprise one or more of Lecithin, for example sunflower lecithin, Polysorbate 80, hydrogenated Castor Oil, and sodium lauroly lactylate.

The emulsifiers may comprise one or more of sodium lauroly lactylate, Polyglycerol Lauroyl Lactylate, and Sodium Stearoyl Lactylate or combinations thereof. Beneficially sodium lauroly lactylate is solid at room temperature and may increase the delivery of and or release of Lauric acid which may have some similar attributes to 2,3 dihydroxypropyl dodecanoate. It is desirable to include in a composition of the invention an emulsifier having a hydrophobic to lipophilic balance (HLB) of greater than 12. Beneficially sodium lauroyl lactylate, Polyglycerol Lauroyl Lactylate, and Sodium Stearoyl Lactylate have a hydrophobic to lipophilic balance (HLB) of greater than 12.

The emulsifiers may comprise short chain monoglycerides, for example short chain monoglycerides with a (carbon) chain length of 10 or less, for example a (carbon) chain length of 3, 4, 8, and/or 10. Beneficially short chain monoglycerides may act as a solvent and help stabilise liquid dosage formulations. Beneficially short chain monoglycerides may act as a competitor for lipase to prevent lipase degradation of 2,3 dihydroxypropyl dodecanoate in the digestive system. Beneficially short chain monoglycerides may be bioactive themselves and may act as an anti-microbial in the digestive tract. Short chain monoglycerides which enter the blood stream are metabolised in the liver.

The emulsifier of the invention may comprise lecithin. Beneficially lecithin may act as both an emulsifier and a taste masking agent to make the composition more palatable.

The emulsifiers may comprise a combination of one or more polysorbates, for example polysorbate 80, one or more short chain monoglycerides, for example one or more short chain monoglycerides with a chain length of 10 or less, and/or lecithin.

Beneficially all the components of the composition of the present invention are Generally Recognised as Safe (GRAS) by the Food and Drug Administration of the United States of America. For example the emulsifier of the present invention is GRAS.

The composition of the invention may comprise additional emulsifiers. The additional emulsifiers may be selected from the group of short chain monoglycerides, short chain fatty acids, glyceryl polyethyleneglycol ricinoleate, and sucroglycerides or combinations thereof.

The composition of the invention may also further comprise additional emulsifiers such as plant extracts, for example extracts of Quillaia, Yucca or seaweed. Beneficially plant extracts which have additional bioactive properties in addition to emulsifying properties are preferred.

The composition of the invention may also further comprise plant extracts with bioactive properties which do not have emulsifying properties.

The composition of the invention may also further comprise plant extracts which act as flavouring agents, for example berry extracts.

The composition of the invention may also further comprise plant extracts which act as colouring agents, for example berry extracts, Redbeet (Beta Vulgaris), Elderberry (anthocyanin), Natural Carotene, Purple Sweet Potato, Chlorophyll, Annatto, Lutein, Paprika, and/or Turmeric.

The composition of the invention may further comprise taste masking agents, for example sweetness enhancers such as sorbitol and/or sucralose.

The composition of the invention may further comprise plant extracts which have antimicrobial, for example antiviral activity, antioxidant, and/or anti-inflammatory activity. For example citrus fruit and grape extracts. For example plant extracts as shown in the table below.

| Antiviral activity against | Active compounds | Plant | Mode of action |
|---|---|---|---|
| Influenza A and B virus | 3-O-rutinosides and 3-O- | Blackcurrant berries | Inhibition of virus binding to |
| | glucosides of malvidin, | (Ribes nigrum) | cells. Also, removal of virus |
| | pelargonidin, peonidin, | | from infected cells |
| | petunidin, delphinidin, | | |
| | cyanidin, and cyanidin 3-O- | | |
| | arabinoside | | |
| Influenza virus | Cyanidin-3-sambubiocide | Black elderberry | Inhibition of viral mutation |
| | | (Sambucus nigra) | and preventing adaptation |
| | | | to the antiviral action of |
| | | | anthocyanins |
| HSV-1 (herpes simplex | Total anthocyanins | Strawberries (Fragaria × | Decrease activity of HSV-1 |
| virus-1) | | ananassa) | virus |
| Adenovirus 36 | Total extract | Mulberry (Morus alba) | Inhibition of virus |
| | and kuromanin chloride | | replication |
| Simian rotavirus SM-11 | Total anthocyanins | Cranberry (Vaccinium | Inhibition of viral adsorption |
| | | macrocarpon) | of phage T4 and replication |
| | | | of rotavirus |
| Influenza A virus | Total anthocyanins | Wolfberry | Weaken the inflammatory |
| | | (Lycium barbarum) | cytokines in the lungs and |
| | | | rise of T cells |
| Influenza A virus, | Total anthocyanins | Chokeberry fruits | Inhibition of Influenza virus |
| Adenovirus and human | | (Aronia melanocarpa) | in its initial stage |
| immunodeficiency virus | | | |
| (HIV-1) | | | |

In the composition of the invention the 2,3 dihydroxypropyl dodecanoate may be encapsulated in the emulsifier. Beneficially encapsulation of the 2,3 dihydroxypropyl dodecanoate in the emulsifier may protect the 2,3 dihydroxypropyl dodecanoate from lipases in the gastrointestinal tract. Beneficially encapsulation of the 2,3 dihydroxypropyl dodecanoate may enhance absorption of 2,3 dihydroxypropyl dodecanoate in the gastrointestinal tract. Beneficially encapsulation of the 2,3 dihydroxypropyl dodecanoate may enhance distribution of 2,3 dihydroxypropyl dodecanoate throughout the gastrointestinal tract.

In the composition of the invention the emulsifier may be encapsulated in the 2,3 dihydroxypropyl dodecanoate. Beneficially encapsulation of the emulsifier in 2,3 dihydroxypropyl dodecanoate may protect the 2,3 dihydroxypropyl dodecanoate from lipases in the gastrointestinal tract. Beneficially encapsulation of the emulsifier in 2,3 dihydroxypropyl dodecanoate may enhance absorption of 2,3 dihydroxypropyl dodecanoate in the gastrointestinal tract. Beneficially encapsulation of the emulsifier in 2,3 dihydroxypropyl dodecanoate may enhance distribution of 2,3 dihydroxypropyl dodecanoate throughout the gastrointestinal tract.

The composition of the invention may be in liquid form, for example liquid oral dosage form, for example wherein the 2,3 dihydroxypropyl dodecanoate is in particulate (solid) form. Beneficially when the composition of the invention is in liquid form it allows for rapid dissolution and uptake in the gastrointestinal tract. Beneficially when the composition of the invention is in liquid form it allows for rapid dissolution and biodistribution throughout the gastrointestinal tract. Beneficially the composition in liquid form enhances bioavailability of the 2,3 dihydroxypropyl dodecanoate. The composition in liquid form may inhibit or compete with the action of lipase on 2,3 dihydroxypropyl dodecanoate in the gastrointestinal tract preventing lipase from cleaving 2,3 dihydroxypropyl dodecanoate to dodecanoic acid and glycerol. The composition in liquid form may allow for rapid dissolution and uptake of 2,3 dihydroxypropyl dodecanoate in the gastrointestinal tract. The composition in liquid form may increase the amount of 2,3 dihydroxypropyl dodecanoate absorbed via the lymphatic system, absorption through the lymphatic system may allow the 2,3 dihydroxypropyl dodecanoate to avoid the liver where it may be broken down to metabolites and energy.

The composition of the invention may be formulated in a liquid (oral) dosage form. The components of a representative composition when in liquid dosage form are according to the invention are listed in Table 1:

TABLE 1

| | Wt % (based on the total |
|---|---|
| Material | weight of the composition) |
| 2,3 dihydroxypropyl dodecanoate | 8-20% |
| Lysolecithin | 4-10% |
| Water | 0.5-50% |
| Lactic Acid white 80% | 0-7% |
| Glyceryl polyethyleneglycol | Up to 40% |
| ricinoleate (Bredol 693) | |
| E474 | Up to 40% |
| Glycerol monopropionate | 0-10% |
| Glycerol monobutyrate | 0-12% |

The composition in liquid dosage form may comprise from 0.001 wt % to 20 wt % of 2,3 dihydroxypropyl dodecanoate based on the total weight of the composition, for example 5 wt % to 15 wt %, for example 5 wt % to 10 wt %, for example 7 wt % to 15 wt %, for example 7 wt % to 10 wt %. A composition comprising more than 20 wt % of 2,3 dihydroxypropyl dodecanoate may not be liquid and as such may not form a liquid dosage form. It will be appreciated that a composition comprising more than 20 wt % of 2,3 dihydroxypropyl dodecanoate may be liquid with the inclusion of organic solvents, such as ethanol, which may be considered intoxicating, hazardous or unpalatable. Preferably the composition does not comprise organic solvents such as ethanol. The composition in liquid dosage form comprising less than 0.001 wt % may not provide an adequate dose of 2,3 dihydroxypropyl dodecanoate. It will be appreciated that a minimum dosage of about 1 g of 2,3 dihydroxypropyl dodecanoate is required to meet thresholds of bioavailability in a typical adult and the liquid dosage form, for example in a beverage, may be any suitable wt % based on the total weight of composition provided the dosage of 2,3 dihydroxypropyl dodecanoate is 1 g or higher.

The composition in liquid dosage form may comprise from 40 wt % to 80 wt % water based on the total weight of the composition, for example 50 wt % to 70 wt %.

The composition in liquid dosage form may comprise 4 wt % to 10 wt % based on the total weight of the composition.

The composition of the invention may be in colloidal form, for example wherein the 2,3 dihydroxypropyl dodecanoate is in particulate (solid) form. When the composition of the invention is in colloidal form the 2,3 dihydroxypropyl dodecanoate may be in a colloid suspension optionally encapsulated in the emulsifier.

The composition in the invention may be in a liquid form and/or a semi-solid form, optionally encapsulated as soft gels.

The composition of the invention may be in solid form. Beneficially when the composition of the invention is in solid form it allows for rapid dissolution and uptake in the gastrointestinal tract. Beneficially when the composition of the invention is in solid form it allows for rapid dissolution and uptake in and biodistribution throughout or within specific regions of the gastrointestinal tract. Beneficially the composition in solid form enhances bioavailability of the 2,3 dihydroxypropyl dodecanoate. The composition in solid form may inhibit or compete with the action of lipase on 2,3 dihydroxypropyl dodecanoate in the gastrointestinal tract preventing lipase from cleaving 2,3 dihydroxypropyl dodecanoate to dodecanoic acid and glycerol. The composition in solid form may allow for rapid dissolution and uptake of 2,3 dihydroxypropyl dodecanoate in the gastrointestinal tract. The composition in solid form may increase the amount of 2,3 dihydroxypropyl dodecanoate absorbed via the lymphatic system, absorption through the lymphatic system may allow the 2,3 dihydroxypropyl dodecanoate to avoid the liver where it may be broken down to metabolites and energy.

The composition of the invention may exhibit modified biodistribution throughout or within specific regions of the gastrointestinal tract. A composition of the invention may have modulatory effects, including health promoting effects through direct or indirection actions on the gastrointestinal microbiome. For example, an effect on host-microbiome interactions that contribute to the pathophysiology of chronic diseases, caused by, amongst other entities, microbe produced short-chain fatty acids, host-microbe co-metabolites such as bile acids and trimethylamine N-oxide (TMAO) and host related molecules, including, but not limited to gut hormones, inflammatory cytokines, mediators of intestinal barrier function. It is known that antibiotic-caused changes in intestinal flora (dysbiosis) can have various effects on the host. Secondary bile acids produced by intestinal bacteria are ligands for specific nuclear receptors such as but not limited to FXR agonists, which regulate glucose, lipid, and drug metabolism in the liver. Changes in secondary bile acids caused by antibiotic-induced dysbiosis on the host physiology, especially glucose, lipid, and drug metabolism. Oral administration of non-absorbable antibiotics for 5 days, decreases amounts of secondary bile acid-producing bacteria in faeces leading to a reduction in secondary bile acid [lithocholic acid (LCA) and deoxycholic acid (DCA)] levels in the liver. Serum glucose and triglyceride levels were also decreased, and these decreases were reversed by LCA and DCA supplementation. Quantitative proteomics demonstrated that the expression levels of proteins involved in glycogen metabolism, cholesterol, bile acid biosynthesis, and drug metabolism (Cyp2b10, Cyp3a25, and Cyp51a1) were altered in the liver in dysbiosis, and these changes were reversed by LCA and DCA supplementation. Thus, it has been suggested that secondary bile acid-producing bacteria contribute to the homeostasis of glucose and triglyceride levels and drug metabolism in the host, and have potential as therapeutic targets for treating metabolic disease. Either through a direct effect of 2,3 dihydroxypropyl dodecanoate or metabolities thereof on hepatic enzymes or an indirect microbiome-induced effect on the production of secondary bile acids, orally administered 2,3 dihydroxypropyl dodecanoate comprising the current invention may contribute to the homeostasis or beneficial modulations of glucose and triglyceride levels and drug metabolism in the host. A further benefit may be a reduced need for antibiotics or other antimicrobials as well as to support the microbiome after use of antibiotics such that the healthy balance is restored.

The composition of the invention may be in solid form wherein the composition is coated or encapsulated in an enteric coating, for example coated or encapsulated in Poly(methacylic acid-co-methyl methacrylate) 1:2. Desirably an enteric coating which helps prevent dissolution of the composition in the gastric environment due to the acidity of the stomach and may be a polymer barrier. Beneficially this may prevent the action of lipase on 2,3 dihydroxypropyl dodecanoate in the gastrointestinal tract preventing lipase from cleaving 2,3 dihydroxypropyl dodecanoate to dodecanoic acid and glycerol. The composition in solid form wherein the composition is coated with an enteric coating may allow for uptake of 2,3 dihydroxypropyl dodecanoate in the gastrointestinal tract. The composition in liquid or semi-solid soft gel form or in solid form coated with an enteric coating may increase the amount of 2,3 dihydroxypropyl dodecanoate available for absorption via the lymphatic system. Absorption through the lymphatic system may allow the 2,3 dihydroxypropyl dodecanoate to avoid the liver where it would otherwise be broken down to metabolites and energy.

The composition of the invention may be formulated as a slow-release formulation.

The composition of the invention may be formulated in solid form with a particle size of less than 1000 microns, for example a particle size of about 200 to about 1000 microns, for example 400 to 800 microns, for example 500 to 700 microns. Beneficially a particle size of less than 1000 microns provides the composition with a good dissolution profile.

The components of a representative composition when in solid dosage form are according to the invention are listed in Table 2:

TABLE 2

| | Wt % (based on the total |
|---|---|
| Material | weight of the composition) |
| 2,3 dihydroxypropyl dodecanoate | 10-60% |
| Calcium dodecanoate | 20-45% |
| Emulsifier (Lysolecithin) | 10-45% |

The composition in solid form may comprise 10 wt % to 45 wt % emulsifier based on the total weight of the composition.

The composition in solid form may comprise from 10 wt % to 30 wt % fillers, for example silica, based on the total weight of the composition.

When the composition of the invention is in solid form the composition of the invention may be a solid stabilised nanoemulsion or solid stabilised micro-emulsion. Alternatively, when the composition of the invention is in solid form the 2,3 dihydroxypropyl dodecanoate may be encapsulated in a liposome, for example a phosphatidylcholine liposome or derivatives thereof, and the liposome acts as the emulsifier. Beneficially the solid form of a composition of the invention may form a microemulsion or a nanoemulsion, for example in fluids with a pH in the range from about 2 to about 8, such as in gastric fluid in the gastrointestinal tract.

The components of a representative composition of the invention when in solid form as a liposome are listed in Table 3:

TABLE 3

| | Wt % (based on the total |
|---|---|
| Material | weight of the composition) |
| 2,3 dihydroxypropyl dodecanoate | 10-20% |
| Emulsifier (Phosphatidylcholine Liposomes) | 80-90% |

The composition of the invention may be an oral dosage anti-microbial agent, for example as an anti-viral, anti-bacterial, anti-fungal and/or anti-protozoan agent. Beneficially the composition of the invention prevents 2,3 dihydroxypropyl dodecanoate, which is a potent anti-microbial, from being broken down into dodecanoate and glycerol which are not effective anti-microbial agents.

A composition of the invention may be liquid above about 55° C. and a solid below 55° C. Beneficially a composition which is liquid above about 55° C. may be suitable for gel filling capsules and solid/semisolid at room temperature, therefore not requiring banding of capsule form. Beneficially a composition which is liquid above about 55° C. may selfemulsify in water at 37° C. within 15 mins. Beneficially a composition which is liquid above about 55° C. may be formulated as gastric resistant capsules and may have gastric lipase avoidance and proximal intestinal release.

A composition of the invention may be liquid above about 55° C. and a solid below 55° C. may comprise 40 wt % to 95 wt % 2,3 dihydroxypropyl dodecanoate based on the total weight of the composition.

A composition of the invention may be liquid above about 55° C. and a solid below 55° C. may comprise 5 wt % to 60 wt % emulsifier based on the total weight of the composition. For example, the emulsifier may comprise one or more of 5 wt % to 60 wt % Sodium Lauroyl Lactylate and 5 wt % to 60 wt % polysorbate 80 based on the total weight of the composition. For example the composition may comprise one or more of 25 wt % to 45 wt % Sodium Lauroyl Lactylate and 10 wt % to 20 wt % polysorbate 80 based on the total weight of the composition.

The composition of the invention may be coated with a polysaccharide, for example maltodextrin or inulin. Beneficially coating the composition of the invention causes the composition to be released in the colon of the GI tract where lipases may not be present. Beneficially coating the composition of the invention causes the composition to be released in the colon of the GI tract where released 2,3 dihydroxypropyl dodecanoate or metabolites thereof may exert health-promoting modulatory effects on the microbiome, such actions including, but being limited to modulating the balance of microbial strains and or the production of short chain fatty acids, including butyrate and derivatives thereof as well as lineoic acid as well as hydrogen forms and other gases, such as, but not limited to, carbon dioxide.

The composition of the invention may be a broad spectrum anti-microbial agent. That is the composition of the invention is effective against a wide range of microbial infectious agents. For example, the composition of the invention is effective against lipid enveloped viruses such as coronaviruses.

The composition of the invention wherein a single oral dose of the composition at a dosage of 0.025 g/kg body weight causes blood plasma levels of 2,3 dihydroxypropyl dodecanoate to exceed 3.9 micrograms/ml peak concentration within 6 hours.

The composition of the invention wherein dosing of the composition at a dosage of 0.025 g/kg body weight every 12 hours causes extracellular fluid in the lungs to have a concentration of 2,3 dihydroxypropyl dodecanoate which exceeds 3.9 micrograms/ml peak after 92 hours.

The composition of the invention may further comprises a further pharmaceutically active agent such as an analgesic, antipyretic such as paracetamol, anti-inflammatories, additional antimicrobial agents, including anti-viral molecules, and/or orally or otherwise administered vaccines, including live-attenuated vaccines administered orally.

The composition of the invention may be microstatic and/or microcidal. A microstatic composition prevents microbial growth. A microcidal composition actively kills microbes.

The composition of the invention may further comprise one or more modulators of Interleukin 6 (“IL-6”), for example naturally occurring modulators such as those found in lithothamnion algae such as lithothamnion seaweed, plant extracts, minerals and/or prebiotics.

The composition of the invention may further comprise one or more nutraceuticals such as vitamin C (ascorbic acid) and/or zinc.

The composition may further comprise one or more vitamins, for example water soluble and/or fat-soluble vitamins, preferably the vitamins are fat soluble vitamins. For example, the composition may comprise the fat-soluble vitamins, Vitamin A, D, E, and/or K. Preferably the composition comprises vitamin D, preferably in the form of vitamin D3.

The composition may further comprise one or more minerals, for example calcium, magnesium, sodium, chloride, potassium, selenium, iron, zinc, phosphorus, iodine, copper, fluoride, molybdenum, manganese, and/or chromium.

The composition may further comprise one or more of selenium, for example selenium yeast, beta glucan, docosahexaenoic acid, eicosapentaenoic acid, vitamin B3 and derivatives, for example Nicotinamide riboside and Nicotinamide mononucleotide, vitamin B6, for example Pyridoxal-5-Phosphate, vitamin B12, for example methylcobalamin powder, folic acid, for example Pteroyl Glutamic Acid. Beneficially Vitamin B3 and derivatives, for example Nicotinamide riboside and Nicotinamide mononucleotide, may help reduce severity and duration of respiratory viral disease.

The composition may further comprise one or more of sleep and/or mood enhancers, for example Melotonin, Ginkgo biloba, Glycine; energy enhancers, for example Q10, Ubiquinol Acetate, Pyrroloquinoline quinone disodium salt [PQQ], Ginsing, Actamide with Beta-cyclodextrin base; Gut health enhancers, for example Lecihtin, Mannan/Fructo oligosaccharides, Glutamine; Pain & Inflammation reduction agents, for example Curcumin, DHA/EPA, Boswellia serrata, Hyaluronic acid; Heart health enhancers. for example, Coenzyme Q10, DHA/EPA, lactotripeptide, Beta Glucan (Oat); and/or cognitive enhancers, for example Selenium, Lecihtin, Ginkgo biloba.

The composition may further comprise one or more of omega oils, for example omega oil 3, 6, or 9; antioxidants such as Coenzyme 10 (CoQ10), for example ubiquinone, ubisemiquinone, ubiquinol; members of the vanillanoid family, for example capsaicin or resiniferatoxin; and compounds which may have anti-inflammatory, antioxidant & immunity activity, for example ginger, turmeric, black pepper, cardamon, fennel (essential oil), Ashwagandha, Guduchi, Long Pepper, Shatavri, Indian Ipedac, Coleus, Basil, Liquorice, Nigellin, Drumstick Tree, and Neem.

In another aspect the invention also relates to a nutraceutical composition comprising the composition of the invention.

In another aspect the invention also relates to a food composition comprising the composition of the invention.

In another aspect the invention also relates to a beverage composition comprising the composition of the invention, for example smoothies, juices, sports and performance products, or baby milk formula.

The composition of the present invention may be used as an anti-microbial agent, for example as an anti-viral, anti-bacterial, anti-fungal and/or anti-protozoan agent optionally in an oral dosage form.

The composition of the invention may be effective for use in the treatment of respiratory tract infections for example in mammals and birds. Surprisingly a composition comprising 2,3 dihydroxypropyl dodecanoate and an emulsifier can be used in the treatment of respiratory tract infection in mammals. Beneficially the composition of the invention increases the bioavailability of 2,3 dihydroxypropyl dodecanoate to an effective therapeutic dose for respiratory tract infections. A surprising attribute of the liquid, emulsion-based formulations of the invention is a mouth feel as well as an induced nasal and throat (pharynx and larynx) sensation attributed to viscosity, pH and other features of the invention. It is known that several infectious agents entering the body via the mouth or nasal passage may reside and replicate in the naso-pharynx-larynx region. Beneficially, the residence time and biodistribution of 2,3 dihydroxypropyl dodecanoate or metabolites thereof as well as other molecules, including vitamins and minerals, in the oro-naso-pharynx-larynx region may contribute to anti-pathogenic effects, including viruses, bacteria and fungi, and outcomes.

The composition of the invention may be used to treat lower respiratory tract infections, for example pneumonia, bronchitis, bronchiolitis, or influenza. Surprisingly a composition comprising 2,3 dihydroxypropyl dodecanoate and an emulsifier can be used in the treatment of lower respiratory tract infection. Beneficially the composition of the invention increases the bioavailability of 2,3 dihydroxypropyl dodecanoate to an effective therapeutic dose for lower respiratory tract infections.

The composition of the invention may be used in the treatment of respiratory tract infections in pigs, for example influenza and/or meningitis and/or porcine reproductive and respiratory syndrome (PRRS). Surprisingly a composition comprising 2,3 dihydroxypropyl dodecanoate and an emulsifier can be used in the treatment of respiratory tract infections in pigs. Beneficially the composition of the invention increases the bioavailability of 2,3 dihydroxypropyl dodecanoate to an effective therapeutic dose for respiratory tract infections in pigs.

The composition of the invention may be used in the treatment of respiratory tract infections in cattle, for example Respiratory Syncytial Virus (RSV) and/or Parainfluenza Virus 3 (PI3). Surprisingly a composition comprising 2,3 dihydroxypropyl dodecanoate and an emulsifier can be used in the treatment of respiratory tract infections in cattle. Beneficially the composition of the invention increases the bioavailability of 2,3 dihydroxypropyl dodecanoate to an effective therapeutic dose for respiratory tract infections in cattle.

The composition of the invention may be used in the treatment of respiratory tract infections in humans, for example influenza and/or coronavirus infection, including COVID-19, in humans. Surprisingly a composition comprising 2,3 dihydroxypropyl dodecanoate and an emulsifier can be used in the treatment of respiratory tract infections in humans. Beneficially the composition of the invention increases the bioavailability of 2,3 dihydroxypropyl dodecanoate to an effective therapeutic dose for respiratory tract infections in humans.

The composition of the invention may be used in the treatment of avian microbial infection, for example influenza in poultry.

The composition of the invention may be used for the treatment of microbial infection in fish, for example early mortality syndrome in shrimp. Beneficially the composition of the invention may be used in treating microbial infection in fish for example those which have relatively short digestive retention times.

In another aspect the invention relates to a method of treatment of infectious disease such as respiratory tract infections in an animal subject such as a human subject in need thereof, comprising administering to the subject the compound 2,3 dihydroxypropyl dodecanoate.

In another aspect the invention relates to a method of treatment of infectious disease such as respiratory tract infections in an animal subject such as a human subject in need thereof, comprising administering to the subject the composition of the invention.

In another aspect the invention relates to a method of modulating triglyceride metabolism, for example high density lipoprotein and/or low-density lipoprotein metabolism, in an animal subject such as a human subject in need thereof, comprising administering to the subject the composition of the invention. Beneficially administering the composition of the invention to a subject may change the HDL:LDL ratio to provide beneficial cardiovascular, inflammatory and other health-promoting effects.

In another aspect the invention relates to a method of modulating glucose metabolism in an animal subject such as a human subject in need thereof, comprising administering to the subject the composition of the invention.

The invention may be useful in providing benefit in restoration of the gut microbiome following use of antibiotics and/or other antimicrobials or other factors, such as chemotherapy or radiation therapy that may alter the gut microbiome and induce dysbiosis within the gut.

The invention may reduce the need for antibiotics and other antimicrobials to reduce the risk of dysbiosis and/or the induction of antimicrobial resistance.

The invention may enhance gastrointestinal, including epithelial barrier, function.

In another aspect the invention relates a method of reducing Bilirubin levels in an animal subject such as a human subject in need thereof, comprising administering to the subject the composition of the invention. Bilirubin is a yellowish pigment that is made during the normal breakdown of red blood cells. Bilirubin passes through the liver and is eventually excreted out of the body. Higher than normal levels of bilirubin may indicate different types of liver or bile duct problems. Occasionally, higher bilirubin levels may be caused by an increased rate of destruction of red blood cells (hemolysis). Reducing bilirubin levels may have beneficial health effects.

In another aspect the invention relates to a method of increasing alkaline phosphate levels in an animal subject such as a human subject in need thereof, comprising administering to the subject the composition of the invention. Alkaline phosphatase functions as a host defence molecule and is present in many cells and organs (e.g. intestine, placenta, liver, kidney and bone). Alkaline phosphatase has a dual mode of action. First, it binds to and, subsequently, dephosphorylates lipopolysaccharide (LPS). Second, the enzymatic reaction product monophosphoryl-LPS is a non-toxic substance for mammals which acts as a partial antagonist on the LPS receptor complex. In several animal studies, administration of alkaline phosphatase attenuates the inflammatory response and reduces mortality. Increasing alkaline phosphate levels may have beneficial health effects.

In another aspect the invention relates to a method of decreasing urea levels in an animal subject such as a human subject in need thereof, comprising administering to the subject the composition of the invention. Beneficially a reduction in urea levels may reduce risk or symptoms of uremia, for example gout and/or gouty arthritis.

The preferred route of delivery of the composition of the invention is by oral administration. The composition of the invention may be taken by itself. The composition of the invention may be mixed with food. The composition of the invention may be mixed with a liquid. The composition of the invention may be administered in enteral form such as by tube feeding.

A mouthwash may be used to deliver the composition of the invention to the oral cavity, nasal cavity, pharynx and/or larynx to confer protection to the oral, nasal, pharynx and/or larynx. The mouthwash may be dispensed following gargling or swallowed for systemic and/or gastrointestinal effects.

A suppository or enema form for administration such as rectal administration, or pessary, wash or other format for administration such as vaginal administration may be used to deliver the composition of the invention. Such forms may provide beneficial local, topical effects and/or permit systemic absorption.

The bioavailability of 2,3 dihydroxypropyl dodecanoate in the composition of the invention is enhanced by the emulsifier. Enhanced bioavailability of 2,3 dihydroxypropyl dodecanoate will have benefits in animal and human health as an anti-viral and general anti-microbial agent. The enhanced bioavailability provides the composition of the invention with excellent antimicrobial, such as antibacterial and/or antiviral, efficacy. The enhanced bioavailability allows for meaningful studies of pharmacodynamics, pharmacokinetics, safety and effectiveness of the molecule. Of particular interest are systemic and respiratory infections for which unenhanced dosage forms would be demonstrably less effective. For example, lipid enveloped viruses, such as, Influenza viruses, corona viruses, RS viruses, Herpes simplex viruses, Hepatitis Viruses, HIV, Gram positive bacterial infections, fungal infections and general broad-spectrum applications.

The composition of the invention may be packaged in packaging formats which may support shelf-life and other attributes of the composition. For example, the composition may be packaged in containers comprising PET, HDPE, glass and/or metal. For example, the composition may be packaged in sachets or pouches, for example Kraft, aluminium foil/metalized films, transparent/metalised laminates, which may be biodegradable and/or compostable. These packaging formats may be suitable wherein the composition is in an emulsion or granule form.

The composition of the invention may be formulated in capsule form, for example hard gelatine capsules, Hydroxypropyl Methylcellulose capsules, Poly(methacylic acid-co-methyl methacrylate) 1:2 capsules, soft gelatine capsules, liquid filled hard capsules, softgels, semi-solid filled hard or soft gel capsules, enteric capsules and/or delayed, sustained or targeted release capsules.

Following administration of 2,3 dihydroxypropyl dodecanoate in emulsion form to large animals infected with respiratory viruses, the beneficial effects observed was superior to non-formulated 2,3 dihydroxypropyl dodecanoate in powder form. Dissolution of 2,3 dihydroxypropyl dodecanoate formulated in emulsion form is rapid and thorough in acid and neutral pH media, with or without added surfactant.

In a further aspect the invention provides a method of modulating gastrointestinal microbiota in an animal subject such as a human subject in need thereof, comprising administering to the subject the composition of the invention. The compositions of the present invention may contribute to the diet to alter the gut microbiome, and in turn, the crosstalk among secondary metabolic pathways; promote the interdependence between the amount of dietary fat, the fatty acid composition, may be dependent on the effects of timing, status (fed and/or fasted) and route of administration on gut microbiota community, and the impact of microbiota-derived fatty acids; effect the bile acid composition, and modulate the role of bile acids on the gut microbiota; as well as impact endogenous and exogenous intestinal micronutrients and metabolites; the above modulations may have impact not only on gut health, but also impact systemic health.

In the composition of the invention complete dissolution of 2,3 dihydroxypropyl dodecanoate may occur within 7 hours, for example within 4 hours, for example 2 hours, for example within 1 hour, for example within 30 minutes in a pH-independent manner.

In the composition of the invention which further comprises vitamin D, for example vitamin D3, complete dissolution of 2,3 dihydroxypropyl dodecanoate and effective systemic absorption of vitamin D, for example vitamin D3 occurs within 12 hours, for example within 6 hours, for example 2 hours.

The composition of the invention may be suitable for administration in the fed or fasted state.

### DETAILED DESCRIPTION OF THE INVENTION

The following examples are not intended to limit the present application.

### Examples

1800 pigs weighing from 40 to 50 kg body weight (BW) were diagnosed with both porcine reproductive and respiratory syndrome (PRRS) and influenza positive (Human variant swine flu H1N1) concurrently. The pigs were showing clinical signs of fever, coughing, poor appetite and high mortality.

The pigs were randomly assigned to three groups:

1) Positive control group (600 pigs) received 0.08 g/kg BW of a commercial formulation of 2,3 dihydroxypropyl dodecanoate on a silica carrier in feed;

2) LDF1 low dose group (600 pigs) received 0.015 g/kg BW of a composition of the invention in liquid form comprising 6.5 wt. % 2,3 dihydroxypropyl dodecanoate (based on the total weight of the composition) and an emulsifier in feed;

3) LDF1 High dose group (600 pigs) received 0.025 g/kg BW of a composition comprising of the invention in liquid form comprising 6.5 wt. % 2,3 dihydroxypropyl dodecanoate (based on the total weight of the composition) and an emulsifier in feed.

The pigs were monitored daily over 14-day period to assess symptoms on a group basis. A Daily score of symptom severity within each group was rated from 0 to 5 for each group on each day based on 15-minute observation of coughing. Coughing is a symptom of PRRS and influenza. The more pigs who cough means the more animals who currently exhibit symptoms of these diseases and provides a good representation of the overall health of each group.

Score 0 represents numerically no animals coughing that is, no animals from the 600 pigs in each group coughed during the 15 minutes observation. A score of 1 means a single pig in 600 coughed during the 15-minute observation period, a score of 2 score means two pigs coughed during the 15-minute observation period, a score of 3 means three pigs coughed during the 15-minute observation period, a score of 4 means 4 pigs coughed during the 15-minute observation period, a score of 5 means five or more animals coughed during the 15-minute observation period. Daily mortality for each group was recorded. The results are shown in Table 4. As can be seen the pigs who were treated with the composition of the present invention coughed less often. The pigs who were treated with the composition of the invention showed less symptoms of disease and were in overall better heath than those treated with the control composition comprising 2,3 dihydroxypropyl dodecanoate without an emulsifier.

### Results

TABLE 4

| | Control | | | Control | | |
|---|---|---|---|---|---|---|
| | 0.08 g/kg | LDF1 | LDF1 | 0.08 g/kg | LDF1 | LDF1 |
| | (unenhanced) | 0.015 g/kg | 0.025 g/kg | (unenhanced) | 0.015 g/kg | 0.025 g/kg |
| | Symptom | Symptom | Symptom | No. Deceased | No. Deceased | No. Deceased |
| Day | score | score | score | pigs/d | pigs/d | pigs/d |
| 1 | 4 | 4 | 4 | 0 | 0 | 0 |
| 2 | 4 | 4 | 4 | 2 | 1 | 0 |
| 3 | 4 | 2 | 2 | 1 | 0 | 1 |
| 4 | 3 | 2 | 1 | 3 | 0 | 0 |
| 5 | 3 | 1 | 0 | 1 | 0 | 0 |
| 6 | 4 | 1 | 1 | 2 | 1 | 1 |
| 7 | 4 | 1 | 1 | 0 | 1 | 1 |
| 8 | 4 | 0 | 0 | 1 | 0 | 0 |
| 9 | 3 | 1 | 1 | 2 | 3 | 1 |
| 10 | 4 | 2 | 0 | 3 | 2 | 1 |
| 11 | 4 | 3 | 0 | 1 | 2 | 2 |
| 12 | 4 | 1 | 1 | 1 | 1 | 1 |
| 13 | 4 | 1 | 1 | 2 | 0 | 0 |
| 14 | 4 | 1 | 0 | 2 | 0 | 0 |
| AVE | 3.785714 | 1.714286 | 1.142857 | 1.5 | 0.785714 | 0.571429 |

N = 1800 pigs, selected at random to positive control group (n = 600) or LDF1 low dose (n = 600) or LDF1 high dose (n = 600); “pigs/d” is pigs per day.

At the start and end of the trial 5 pigs from the control group and 5 pigs from the LDF1 High dose group were tested for PRRS antibodies by ELISA and presence of PRRS RNA by PCR. In brief, blood was drawn from 5 pigs selected at random from each group on day 1 of the trial. The blood was tested for the presence of PRRS antibodies by ELISA and presence of PRRS RNA by PCR. Blood was drawn from 5 pigs selected at random from each group on day 14 of the trial. The blood was tested for the presence of PRRS antibodies by ELISA and presence of PRRS RNA by PCR. On day 1 all animals tested positive for both PRRS antibodies and PRRS RNA. On day 14 no animal in the treatment group tested positive for PRRS RNA indicating that there was no viral load in these animals that is, the PRRS virus was no longer present in the animals. 4 from 5 animals from the control group tested positive for PRRS RNA indicating that these animals were still infected with the PRRS virus. The results of the testing for PRRS antibodies by ELISA and presence of PRRS RNA by PCR are shown in Table 5.

TABLE 5

| | Control | LDF1 High dose | | LDF1 High |
|---|---|---|---|---|
| | ELISA+/− | ELISA+/− | Control PCR | dose PCR |
| Day 0 | 5/5 positive | 5/5 positive | 5/5 positive | 5/5 positive |
| Day 14 | 5/5 positive | 5/5 positive | 4/5 positive | 0/5 positive |

N = 20 pigs (5 animals selected at random from each group at d 0 and d 14 of trial)

### Open-Label, Randomized, Single-Dose, Five-Period, Crossover, Oral Bioavailability Study in Humans to Evaluate the Biodistribution of 2,3 Dihydroxypropyl Dodecanoate and Vitamin D.

Subjects: 30 health male Healthy male volunteers, as evaluated by medical history, vitals and general clinical examination, of 18 to 45 years (both years inclusive) with BMI of 18.50-29.99 Kg/m² were enrolled. Subjects were evaluated for normal or clinically insignificant biochemical, hematological, urinary, serology, Chest X Ray and ECG values/reports. Subjects were generally healthy as documented by the medical history, physical examination (including but may not be limited to an evaluation of the cardiovascular, gastrointestinal, respiratory, musculoskeletal and central nervous systems) and vital sign assessments. Subjects had no evidence of medical illness during screening and check-in. Screening was performed within 28 days of check in. Subjects had negative urine test for drugs, and negative alcohol breathe analysis. Subjects exhibited no evidence of suspected Covid-19 symptoms. Subjects were excluded based on the following criteria:
History of any major surgical procedure in the past 3 months.
History of any clinically significant cardiac, gastrointestinal, respiratory, hepatic, renal, endocrine, neurological, metabolic, psychiatric, hematological and/or any major surgical procedure in the past three months.
History of chronic alcoholism/chronic smoking/drug of abuse/Hypersensitivity.
Subject who consumed tobacco containing products within 48 hours prior to proposed time of dosing
Present or past history of intake of drugs or any prescription drug or over the counter (OTC) drugs within 7 days which potentially modify kinetics/dynamics of 2,3 dihydroxypropyl dodecanoate or any other medication judged to be clinically significant by the investigator.
Consumption of grapefruit and/or its products within 10 days prior to the start of study.
Subject who had participated in any other clinical study or who had bled during the last 90 days.
Subjects who are allergic to coconut/coconut containing foods or known hypersensitivity to 2,3 dihydroxypropyl dodecanoate/lauric acid or its derivatives
History of difficulty in swallowing.
High blood pressure and asthma
Renal or liver impairment
Subjects, who consumed raw coconut, coconut oil, coconut containing products, etc. in last 5 days were excluded from the study because coconut is a natural source of 2,3 dihydroxypropyl dodecanoate.
History of past and present COVID-19 infection

Study Design

Subjects were randomly assigned to 5 cohorts with six subjects per cohort. The dosing schedule for each cohort is shown in Table 6. A 7-day washout period was provided between dosing. The formulations are described in Table 7. The dose strength of 2,3 dihydroxypropyl dodecanoate was 1.1 grams delivered orally in either powder form (R, C), liquid form (T1) granule form (T2), or granules in an enteric capsule (T3). The components of each formulation are listed in Table 8a and 8b. Reference formulation (R) is 2,3 dihydroxypropyl dodecanoate which is not in combination with an emulsifier. Comparator (C) formulation, Lauricidin®, is an example of a commercially available 2,3 dihydroxypropyl dodecanoate (monolaurin) powder formulation and is available from Med-Chem Labs, AZ, USA. T1 is a liquid dosage formulation comprising 2,3 dihydroxypropyl dodecanoate and an emulsifier. T2 is 2,3 dihydroxypropyl dodecanoate and an emulsifier in a solid form which is a granule form. T3 is 2,3 dihydroxypropyl dodecanoate and an emulsifier in a solid form which is a granule form encapsulated in an enteric capsule. Doses of each formulation were administered according to the dosing method in Table 9.

Dosing was performed after overnight fasting of at least 10.00 hours, in the morning a single oral dose of test product (T1, T2, T3, R or C) were administered (according to the randomization schedule and a minimal coconut free food was given prior to dosing) with final volume of 240±02 mL of water at ambient temperature, to the subjects, in seated upright posture, under the supervision of Investigator/medical officer. Compliance with dosing was confirmed by mouth check of the subjects with the help of tongue depressor and torch light to assess compliance to dosing. Subjects remained seated in upright position for at least 04.00 hours of post dose in each period and only necessary movement will be allowed during this period. Subjects were not allowed to lie down (except as directed by the physician secondary to adverse events) during this restriction period. Thereafter, subjects were allowed to ambulate freely during the remaining period of the study. The subjects did not take part in any strenuous exercise/activity during the study. Drinking water was restricted at least 01.00 hour prior to dosing until 01.00 hour post-dose (except 240±02 mL of water given during dosing). At all other times, drinking water was provided freely. Standard meals were provided at 04.00, 09.00 and 13.00 hours post-dose.

TABLE 6
Study Design

| Informed | | | | | |
|---|---|---|---|---|---|
| Consent & | 7-day | 7-day | 7-day | 7-day | |
| Screening | Washout | Washout | Washout | Washout | End of |
| Treatment | Period | Period | Period | Period | study |
| (6 subjects | Week | Week | Week | Week | Week |
| per cohort) | 1 | 2 | 3 | 4 | 5 |
| Cohort 1 | T1 | T2 | T3 | R | C |
| Cohort 2 | T2 | T3 | R | C | T1 |
| Cohort 3 | T3 | R | C | T1 | T2 |
| Cohort 4 | R | C | T1 | T2 | T3 |
| Cohort 5 | C | T1 | T2 | T3 | R |

TABLE 7

| | | | | Colour/ | Storage |
|---|---|---|---|---|---|
| Formulation | Treatment | Dose Strength | Form | Shape | condition |
| Reference (R) | 2,3 dihydroxypropyl | 1.1 Gram ± 1% | Granule | White | 15° to 25° C. |
| | dodecanoate | | | Granule | |
| | granules | | | | |
| Comparator (C) | 2,3 dihydroxypropyl | 1.1 Gram ± 1% | Granule | White | 15° to 25° C. |
| | dodecanoate | | | Granule | |
| | Lauricidin ® | | | | |
| Test (T1) | Liquid | 15 ml equivalent to | Solution | Purple | 15° to 25° C. |
| | formulation | about 1.1 grams of | | coloured | |
| | containing 2,3 | 2,3 dihydroxypropyl | | solution | |
| | dihydroxypropyl | dodecanoate | | | |
| | dodecanoate | | | | |
| | Solution (7.5%) & | | | | |
| | emulsifier | | | | |
| Test (T2) | Solid dosage | 2.4 Grams ± 1% of | Granule | White | 15° to 25° C. |
| | formulation | granules equivalent | | granule | |
| | containing | to about 1.1 grams | | | |
| | granules of 2,3 | of 2,3 dihydroxypropyl | | | |
| | dihydroxypropyl | dodecanoate | | | |
| | dodecanoate & | | | | |
| | emulsifier | | | | |
| Test (T3) | Enteric dosage | 4 Enteric Capsules | Granule- | 00 size | 15° to 25° C. |
| | formulation | containing 600 mg | filled | White | |
| | containing | granules, | Capsule | capsule | |
| | granules of 2,3 | equivalent to a | | | |
| | dihydroxypropyl | total of about 1.1 | | | |
| | dodecanoate & | grams of 2,3 | | | |
| | emulsifier | dihydroxypropyl | | | |
| | | dodecanoate | | | |

TABLE 8a

| Formulation | Treatment | Components | Analytical Properties |
|---|---|---|---|
| Reference (R) | 2,3 dihydroxypropyl | 2,3 dihydroxypropyl | >90% 2,3 |
| | dodecanoate granule | dodecanoate | dihydroxypropyl |
| | | | dodecanoate |
| Comparator (C) | 2,3 dihydroxypropyl | 2,3 dihydroxypropyl | >90% 2,3 |
| | dodecanoate (Lauricidin ®) | dodecanoate | dihydroxypropyl |
| | | | dodecanoate |
| Test (T1) | Liquid formulation containing | Water, Mono and Di- | 2,3 dihydroxypropyl |
| | 2,3 dihydroxypropyl | Glycerides (2,3 | dodecanoate: 1.1 g |
| | dodecanoate Solution (7.5%) & | dihydroxypropyl | Zinc: 10 mg |
| | emulsifier | dodecanoate), Emulsifiers | Vitamin C: 80 mg |
| | | (Sunflower Lecithin, | Vitamin E: 12 mg |
| | | Polysorbate 80, | Vitamin D3: 25 μg |
| | | Hydrogenated Castor Oil), | Vitamin B12: 2.5 μg |
| | | Sweeteners (Sorbitol, | |
| | | Sucralose), Mixed Berry | |
| | | Extract, Acidity Regulator | |
| | | (Citric Acid, Lactic Acid), | |
| | | Flavours, Natural | |
| | | Flavours, Vitamin C (L- | |
| | | ascorbic acid), Vitamin E | |
| | | (Tocopherols), Zinc | |
| | | Gluconate, Vitamin D3 | |
| | | (Cholecalciferol), Vitamin | |
| | | B12 (Cyanocobalamin) | |
| Test (T2) | Solid dosage formulation | Mono and Di-Glycerides | 2,3 dihydroxypropyl |
| | containing granules of 2,3 | (2,3 dihydroxypropyl | dodecanoate: 1.1 g |
| | dihydroxypropyl dodecanoate | dodecanoate), Emulsifier | Vitamin D3: 2000 IU |
| | & emulsifier | (Sodium Lauroyl Lactylate), | |
| | | Inulin, Vitamin D3 | |
| | | (Cholecalciferol) | |
| Test (T3) | Enteric dosage formulation | Mono and Di-Glycerides | 2,3 dihydroxypropyl |
| | containing granules of 2,3 | (2,3 dihydroxypropyl | dodecanoate: 1.1 g |
| | dihydroxypropyl dodecanoate | dodecanoate), Emulsifier | Vitamin D3: 2000 IU |
| | & emulsifier | (Sodium Lauroyl Lactylate), | |
| | | Inulin, Vitamin D3 | |
| | | (Cholecalciferol), | |
| | | Hypromellose Capsule | |

TABLE 8b

| | R | C | T1 | T2 | T3 |
|---|---|---|---|---|---|
| Component | Weight percent of total composition | | | | |
| 2,3 dihydroxypropyl | >93* | >93* | 7.353 | 45.275 | 45.482 |
| dodecanoate | | | | | |
| Sodium Lauroyl Lactylate | 0.000 | 0.000 | 0.000 | 22.503 | 22.606 |
| Cabosil (silica) | 0.000 | 0.000 | 0.000 | 20.385 | 20.478 |
| Inulin | 0.000 | 0.000 | 0.000 | 10.920 | 10.970 |
| vitamin D 2MIU/g | 0.000 | 0.000 | 0.004 | 0.098 | 0.099 |
| Cabosil (silica) | 0.000 | 0.000 | 0.000 | 0.364 | 0.366 |
| Poly(methacylic acid-co-methyl | 0.000 | 0.000 | 0.000 | 0.455 | 0.000 |
| methacrylate) 1:2 | | | | | |
| Castor oil | 0.000 | 0.000 | 1.470 | 0.000 | 0.000 |
| Polysorbate 80 | 0.000 | 0.000 | 2.940 | 0.000 | 0.000 |
| Monoglyceride blend | 0.000 | 0.000 | 7.353 | 0.000 | 0.000 |
| Chain length 3, 4, 8, 10 | | | | | |
| Vit E 98% DL alphatocopherol | 0.000 | 0.000 | 0.094 | 0.000 | 0.000 |
| Lecitas 4719 | 0.000 | 0.000 | 5.880 | 0.000 | 0.000 |
| Water | 0.000 | 0.000 | 68.120 | 0.000 | 0.000 |
| Ascorbic acid | 0.000 | 0.000 | 0.627 | 0.000 | 0.000 |
| Zinc Gluconate | 0.000 | 0.000 | 0.460 | 0.000 | 0.000 |
| Vit B12 | 0.000 | 0.000 | 0.020 | 0.000 | 0.000 |
| Sorbitol 70% syrup | 0.000 | 0.000 | 3.676 | 0.000 | 0.000 |
| Berry Extract | 0.000 | 0.000 | 1.765 | 0.000 | 0.000 |
| sucralose | 0.000 | 0.000 | 0.196 | 0.000 | 0.000 |
| Total | 100 | 100 | 100 | 100 | 100 |

*the remainder of the composition comprises derivatives of 2,3 dihydroxypropyl dodecanoate such as lauric acid.

TABLE 9

| | |
|---|---|
| Reference (R) | 1.1 ± 1% Grams of powder/granule were weighed in a disposable container and given to |
| | subject for dosing. Subject was requested to swallow/wash down completely with 240 |
| | ml ± 02 ml of water at room temperature. A small volume of water was used for rinsing the |
| | container and the subject drank the rinsed solution. It was ensured that no residue |
| | remained in the disposable container or in the mouth cavity. This was followed by |
| | assessment of Compliance for Dosing. |
| Comparator (C) | 1.1 ± 1% Grams of powder/granule was weighed in a disposable container/spatula and |
| | given to subject for dosing. Subject was requested to swallow/wash down completely |
| | with 240 ml ± 02 ml of water at room temperature. A small volume of water was used |
| | for rinsing the container and the subject drank the rinsed solution. It was ensured that |
| | no residue remained in the disposable container or in the mouth cavity. This was |
| | followed by assessment of Compliance for Dosing. |
| Test (T1) | 15 ml of the liquid dose was measured in a measuring container to which 100 ml of |
| | water was added (total volume 115 ml) in dispensing container, (care was taken to make |
| | sure no sample remained in the measuring cup for each dose), stirred, and given to |
| | subject for dosing. Additional 125 ml ± 02 ml water was added to the same container, |
| | stirred and subject consumed completely ensuring no residue remains. This was |
| | followed by assessment of Compliance for Dosing. |
| Test (T2) | 2.4 ± 1% Grams of granule was weighed in a disposable container/spatula and given to |
| | subject for dosing. Subject was requested to swallow/wash down completely with 240 |
| | ml ± 02 ml of water at room temperature. A small volume of water was used for rinsing the |
| | container and the subject drank the rinsed solution. It was ensured that no residue |
| | remained in the disposable container or in the mouth cavity. This was followed by |
| | assessment of Compliance for Dosing. |
| Test (T3) | 4 Capsules were given to the subject and swallowed with 240 ml ± 02 mL water for |
| | dosing. This was followed by assessment of Compliance for Dosing. |

### Sample Collection

Fecal samples were collected at week 1 and week 2 for all treatment groups during the in-house study period. Fecal sample collected from −12 hrs to 0.00 hrs is considered as pre-dose sample and fecal sample collected after the dosing to 24 hrs is considered as post dose sample. Subject collected stool each time in a labeled disposable container/pack from check in to until 24 hours post dose and stored in the refrigerator (2 to 8° C.). Fecal samples collected from a subject prior the dosing to be pooled, homogenized and transferred in to labeled container. Similarly, all fecal samples collected post dosing from a subject was separately pooled, homogenized and 5 gm of the sample will be transferred into labeled aliquot-I and aliquot-II containers. Aliquots will be stored in a freezer at −70° C.±15° C. until further processing.

Urine samples were collected at week 1 and week 2 for all treatment groups during the in-house period. Urine samples collected from −12 hrs to 0.00 hrs is considered as pre-dose sample and urine sample collected after the dosing to 24 hrs is considered as post dose sample. Subject collected urine sample each time in a labeled disposable container from check in to until 24 hours post dose and stored in the refrigerator (2 to 8° C.). Urine samples collected from a subject prior the dosing to be pooled, mixed and transferred 10 ml into aliquot-I and aliquot-II containers. Similarly, all urine samples collected post dosing from a subject is to be separately pooled, mixed and 10 ml of the sample will be transferred into labeled aliquot-I and aliquot-II containers. Aliquots were stored in a freezer at −70° C.±15° C. until further processing.

Blood samples were collected through an indwelling intravenous cannula placed in a forearm vein. Intravenous indwelling cannula was kept in situ as long as possible by injecting about 0.5 mL of 10 IU/mL of heparin in normal saline solution to maintain the cannula patent for collection of the post-dose samples. In such cases blood samples were collected after discarding the first 0.5 mL of heparinized saline containing blood. The pre-dose sample was collected before dosing and post-dose samples was collected after dosing. The blood samples were collected using syringe and/or adaptor and transferred into pre-labelled K2EDTA vacutainers. Vacutainers were placed upright in a rack kept in wet ice bath until centrifugation and during plasma separation. Blood samples were centrifuged at 4000 RPM for 10 minutes at 02° C. to 08° C. to separate the plasma. Centrifugation was started within 30 minutes of the collection of samples. Plasma samples for vitamin D3 (1 mL Pre-Dose 00.00 Hrs and 1 mL Post Dose 12.00 Hrs) analysis were aliquoted and stored in separate vials.

A total of 17 blood samples were collected per subject per treatment period. The sampling timing is shown in Table 10.

TABLE 10

| | Sample Time Points | Sample Time Points | Volume |
|---|---|---|---|
| S. No | (hours) | (minutes) | of Blood |
| 1 | −12.00 | −720 | 04 mL |
| 2 | −06.00 | −360 | 04 mL |
| 3 | 00.00 | 0 (−20 to 0) | 05 mL* |
| 4 | 00.25 | +15 | 04 mL |
| 5 | 00.50 | +30 | 04 mL |
| 6 | 00.75 | +45 | 04 mL |
| 7 | 01.00 | +60 | 04 mL |
| 8 | 01.25 | +75 | 04 mL |
| 9 | 01.50 | +90 | 04 mL |
| 10 | 01.75 | +105 | 04 mL |
| 11 | 02.00 | +120 | 04 mL |
| 12 | 02.50 | +150 | 04 mL |
| 13 | 03.00 | +180 | 04 mL |
| 14 | 04.00 | +240 | 04 mL |
| 15 | 06.00 | +360 | 04 mL |
| 16 | 12.00 | +720 | 05 mL* |
| 17 | 24.00 | +1440 | 04 mL |

*Additional 1 mL of blood was collected at time-points 00:00 pre-dose and 12:00 post-dose for vitamin D3 analysis.

### Sample Analysis

2,3 dihydroxypropyl dodecanoate in plasma, urine & feces samples and vitamin D3 in plasma was assayed using Liquid Chromatography-Tandem Mass Spectorometry (LC-MS/MS). A solution of internal standard in the concentration of 1 μg/mL for Alpha Monolaurin D5 in Acetone-M: Water (70:30, v/v) was prepared. 50 μL of internal standard solution (Alpha Monolaurin D5 1 μg/mL) to 500 μL of samples. Each sample was analysed on a LC-MS/MS system using a Zorbax XDB C18 (100 mm×4.6 mm, 3.5 μm) column with a mobile phase of Acetone-M: Buffer (90:10 v/v) at a column oven temperature of 40° C. MS/MS was performed with a positive mode of ionization. Chromatograms were acquired using the computer-based Analyst software version 1.6.3, supplied by AB Sciex process data by peak area ratio. The concentration of the unknown is calculated from the following equation using regression analysis of spiked plasma calibration standard with the reciprocate of the drug concentration as a weighting factor (1/X²)

Y=mx+b

Where,

x=Concentration of Alpha Monolaurin

m=Slope of the calibration curve

y=Peak area ratio of Alpha Monolaurin to Alpha Monolaurin D5

b=y−axis intercept of the calibration curve

### Statistical Analysis

Statistical analysis was performed on the pharmacokinetic parameters using SAS®V 9.4. The analysis was performed on data from subjects who complete the entire study.

Summary Statistics, ANOVA, Ratio analysis, Power, Intra subject CV and 90% Confidence Interval were calculated.

Descriptive analysis of plasma concentration (time point wise and formulation wise) and pharmacokinetic parameters—Cmax, AUC0-t, and AUCLast were determined for each test formulations. Calculations include the mean, minimum, maximum, range, standard deviation, Standard error, geometric mean and the coefficient of variation for each PK parameters of 2,3 dihydroxypropyl dodecanoate. Ln-transformed data of Cmax, AUC₀₋ₜ, and AUC_{0-∞} was utilized, when calculating geometric mean and least square ratio.

Intra-subject variability was computed for Ln-transformed parameters Cmax and AUC₀₋ₜ for 2,3 dihydroxypropyl dodecanoate.

The confidence limits are expressed as a percentage of the least square mean (LSM) of the reference formulation. Using the confidence limits of the above confidence interval and the LSM of the reference product, 90% confidence interval for the ratio of the test and reference product means was calculated.

The comparison of interest is Test (T) vs Reference (R). Ratios are in the form: —Test/Reference (T/R). Ratio of means was calculated using the LSM of log-transformed pharmacokinetic parameters (Cmax and AUC₀₋ₜ). Ratio of means is expressed as a percentage of the LSM of the reference formulation.

Ratio of geometric least square means for 2,3 dihydroxypropyl dodecanoate of test (T1, T2, T3) and reference (R) formulations was computed for Ln-transformed pharmacokinetic parameters Cmax, AUC₀₋ₜ, and AUC_{0-∞}.

The power of ANOVA test to detect a 20% mean difference between test formulations was calculated for 2,3 dihydroxypropyl dodecanoate.

### Results

Formulations T1, T2, and T3 comprised vitamin D. Vitamin D (Vitamin D3/Cholecalciferol) was detectable in blood plasma of the subjects after 12 hours post dose. The levels of vitamin D in blood plasma are shown in Table 11. This confirms that the vitamin D of the formulation was bioavailable.

TABLE 11

| 75 μg/3000 IU Vitamin D3 | ng/ml/3000 IU |
|---|---|
| T3 (Capsule) - Vitamin D3 (ng/ml) - Fasted/Light Meal | 4.77 |
| T3 (Capsule) - Vitamin D3 (ng/ml) - Fed/High Fat Meal | 3.37 |
| T2 (Granule) - Vitamin D3 (ng/ml) - Fasted/Light Meal | 5.65 |
| T2 (Granule) - Vitamin D3 (ng/ml) - Fed/High Fat Meal | 7.89 |
| T1 (Emulsion) - Vitamin D3 (ng/ml) - Fasted/Light Meal | 7.34 |
| T1 (Emulsion) - Vitamin D3 (ng/ml) - Fed/High Fat Meal | 8.83 |

The level of 2,3 dihydroxypropyl dodecanoate was determined in urine samples pre and post dosing. The amount of 2,3 dihydroxypropyl dodecanoate was below the level of detection in all samples.

The level of 2,3 dihydroxypropyl dodecanoate was determined in faecal samples pre and post dosing. The amount of 2,3 dihydroxypropyl dodecanoate was below the level of detection in all samples.

The levels of 2,3 dihydroxypropyl dodecanoate was determined in blood plasma at 24 hours post dosing. The mean and maximum levels of 2,3 dihydroxypropyl dodecanoate found in blood plasma are shown in Table 12. Dosing performed with a composition comprising 2,3 dihydroxypropyl dodecanoate and an emulsifier provided elevated blood plasma levels of 2,3 dihydroxypropyl dodecanoate after 24 hours. Increased levels of 2,3 dihydroxypropyl dodecanoate may indicate that the composition comprising 2,3 dihydroxypropyl dodecanoate and an emulsifier has dissolved in the gastrointenstinal tract and has avoided lipases in the gastrointestinal tract which would degrade 2,3 dihydroxypropyl dodecanoate. A composition comprising 2,3 dihydroxypropyl dodecanoate and an emulsifier may form micelles such that it undergoes lymphatic uptake and sustained release from the lymphatic system over time.

TABLE 12

| | Period 4 & 5 combined | | Period 3-5 combined | |
|---|---|---|---|---|
| | Mean | Maximum | Mean | Maximum |
| | ng/ml 2,3 dihydroxypropyl | | ng/ml 2,3 dihydroxypropyl | |
| Treatment | dodecanoate | | dodecanoate | |
| C | 3.376 | 37.139 | 2.185 | 37.139 |
| R | 0 | 0 | 0 | 0 |
| T1 | 23.427 | 167.383 | 15.159 | 167.383 |
| T2 | 212.368 | 2463.105 | 141.579 | 2463.105 |
| T3 | 44.669 | 503.12 | 29.779 | 503.12 |

The health-promoting effect attributed to a wide range of vitamins, minerals and other moieties is well established. The various formulations relating to the present invention are compatible with moieties exhibiting a broad span of solubility profiles, including water soluble vitamins such as Vitamin C as well as oil-soluble vitamins such as Vitamin D. Importantly, human pharmacokinetic data presented herein demonstrate that all formulations evaluated, namely emulsion (T1), enteric coated granules (T2) and enteric capsule (HPMC) encapsulated granules (T3), result in robust systemic absorption following oral administration. The emulsion form (T1) was not associated with a food effect, systemic bioavailability following oral administration of the enteric capsule encapsuled granules (T3) was higher in the fasted than in the fed state while systemic bioavailability following oral administration of the enteric coated granule form (T2) was greater in the fed than in the fasted state. In summary, there is almost no change in Vit D3 differential levels in High fat meal (P5) when compared to Light meal (P4) for Emulsion (T1), such that Vit D3 absorption remained unchanged (or marginally improved) in T1 group when subjects were fed a high fat high calorie meal; There is an increase in Vit D3 differential levels in High fat meal (P5) when compared to Light meal (P4) for Granules (T2), such that Vit D3 absorption increased in T2 group when subjects were fed a high fat high calorie meal; and there is a decrease in Vit D3 differential levels in High fat meal (P5) when compared to Light meal (P4) for Capsules (T3), such that “Vit D3 absorption decreased in T3 group when subjects were fed a high fat high calorie meal.

Correlating the pharmacokinetics observed for Vitamin D3 (Vitamin D), the absorption of other oil soluble molecules, including vitamins, minerals and other moieties, including, but not limited to oil-soluble pharmaceutical agents may be similar. This may include various omega oils (3, 6, 9 or other) as well as antioxidants such as Coenzyme 10 (CoQ10), in any of its three redox states, namely fully oxidized (ubiquinone), semiquinone (ubisemiquinone), and fully reduced (ubiquinol), or members of the vanillanoid family, including, but not limited to capsaicin or resiniferatoxin.

Blood chemistry in all subjects was evaluated pre- and post-dosing in all subjects. As the trial design was a cross over study all subjects had been subject to each formulation.

It was found that there was a statistically significant increase on alkaline phosphatase (p=0.003) in subjects after completion of the study. As it has been observed that administration of alkaline phosphatase attenuates the inflammatory response and reduces mortality, this may contribute to beneficial effects in acute or chronic infection and related conditions, including but not limited to sepsis, septic shock, acute respiratory distress syndrome, acute lung injury, acute kidney injury, acute liver injury and COVID-19.

It was found that there was a statistically significant (p=0.0007) elevation in the levels of HDL detected, with no change in LDL levels (p=0.85), a significant reduction in triglyceride levels (p=0.0127) in subjects upon completion of the study. Importantly, the level of very-low-density lipoprotein (VLDL) cholesterol, produced in the liver and released into the bloodstream to supply body tissues with a type of fat (triglycerides), was significantly lower (p=0.0099). Potential benefits include cardiovascular health, including reduced plaque formation.

It was found that there is a significant reduction in urea levels (p=0.0483) in subjects upon completion of the study. Potential benefits include reducing risk or symptoms of gout and gouty arthritis.

It was found that there was a statistically significant reduction in bilirubin (Direct) (p=0.0001) in subjects upon completion of the study. Potential benefits include treating or preventing liver disease, particularly hepatitis, anemia or certain drug overdoses.

### Dissolution Experiments

The following experiments were performed to compare the dissolution profiles of the formulations C, R, T1, T2, and T3 as shown in Table 8. Dissolution experiments were performed according to the method of (711) Dissolution USP 2016.

Dissolution experiment 1: To compare the dissolution of each formulation in 0.1 N HCL the formulation was added to 0.1 N HCL in 900 ml vessels. The vessels were placed in a basket in a standard dissolution bath at a temperature of 37.5° C. The basket was agitated at 75 RPM for 2 hours. The results are shown in Table 12. Only the liquid dosage form comprising 2,3 dihydroxypropyl dodecanoate and an emulsifier showed dissolution after 2 hours.

TABLE 13

| Sample (n = 6) | Percent of total sample dissolved at 2 hours |
|---|---|
| C (n = 6) | 0 |
| R (n = 6) | 0 |
| T1 (n = 18) | 67.86667 |
| T2 (n = 6) | 0 |
| T3 (n = 6) | 0 |

Dissolution experiment 2: To compare the dissolution of each formulation in pH 7.2 phosphate buffer each formulation was added to pH 7.2 phosphate buffer in 900 ml vessels. The vessels were placed in a basket in a standard dissolution bath at a temperature of 37.5° C. The basket was agitated at 75 RPM for 10 hours. Dissolution was measured at 1, 4, and 7. The results are shown in Table 13. Only the liquid dosage form comprising 2,3 dihydroxypropyl dodecanoate and an emulsifier showed dissolution above 10% after 1, 4, and 7 hours. C is in pellet form from 2 mm to 5 mm in diameter did not dissolve. R is in granule form with a particle size of from 500-1000 microns and without an emulsifier did show dissolution of less than 10% after 4 and 7 hours.

TABLE 14

| | 1 hour | 4 hours | 7 hours |
|---|---|---|---|
| Sample | Percent of total sample dissolved | | |
| C (n = 6) | 0 | 0 | 0 |
| R (n = 6) | 0 | 6.2 | 9.5 |
| T1 (n = 12) | 72.75 | 74.55 | 89.15 |
| T2 (n = 6) | 0 | 0 | 0 |
| T3 (n = 6) | 0 | 0 | 0 |

Dissolution experiment 3: To compare the dissolution of formulation R and T2 in pH 6.8 phosphate buffer containing 0.2% sodium lauryl sulfate (SLS) each formulation was added to 0.1 N HCL in 900 ml vessels. The vessels were placed in a basket in a standard dissolution bath at a temperature of 37.5° C. The basket was agitated at 100 RPM for 6 hours. Dissolution was measured at 2, 4, and 6 hours. The results are shown in Table 14. After 2 hours over 80% of T2 was dissolved indicating greater solubility due to the presence of emulsifiers in the formulation.

TABLE 15

| | 2 hours | 4 hours | 6 hours |
|---|---|---|---|
| Sample | Percent of total sample dissolved | | |
| R (n = 6) | 18.1 | 82.4 | 82.9 |
| T2 (n = 6) | 84.5 | 101.4 | 102.9 |

Dissolution experiment 4: To compare the dissolution of each formulation in pH 7.2 phosphate buffer containing 0.2% sodium lauryl sulfate (SLS) each formulation was added to pH 7.2 phosphate buffer containing 0.2% sodium lauryl sulfate (SLS) in 900 ml vessels. The vessels were placed in a basket in a standard dissolution bath at a temperature of 37.5° C. The basket was agitated at 75 RPM for 7 hours. Dissolution was measured at 1, 4, and 7 hours. The results are shown in Table 15. T2 granules are less soluble than T3 granules in gastric resistant capsule. This indicates an effect of Sustained release coating on the granule. This also indicates minimum effect of gastric resistant capsule in pH 6.8. T1, liquid emulsions are readily soluble, >70% at 1 hour and >95% at 7 hours.

TABLE 16

| | 1 hour | 4 hours | 7 hours |
|---|---|---|---|
| Sample | Percent of total sample dissolved | | |
| C (n = 6) | 0 | 0 | 0 |
| R (n = 6) | 0 | 6.2 | 37.4 |
| T1 (n = 12) | 72.5 | 74.55 | 107.5 |
| T2 (n = 6) | 0 | 0 | 6.2 |
| T3 (n = 6) | 0 | 0 | 37.2 |

Overall, the dissolution experiments show that 2,3 dihydroxypropyl dodecanoate is poorly soluble in acid and intestinal pH range in the absence of extra emulsifiers either incorporated to the formulation or dissolution media. Smaller particle size promotes dissolution of unformulated 2,3 dihydroxypropyl dodecanoate in the presence of SLS in the media. Liquid emulsified formulations have a rapid dissolution profile in acid and intestinal pH ranges. Gastric resistant capsules did not yield 2,3 dihydroxypropyl dodecanoate in acid conditions but did yield >35% at 7 hours in pH 7.2 with SLS in media. Granules without sustained release coating did not yield 2,3 dihydroxypropyl dodecanoate in acid and yielded only 6.2% at 7 hours at pH 7.2 with SLS media, demonstrating a slow-release profile, relative to granules in capsule that were coated with a sustained release coating.

The beneficial effects observed in pig studies for the emulsion form when compared to non-formulated or otherwise sub-optimally formulated 2,3 dihydroxypropyl dodecanoate correlates with the dissolution rate of 2,3 dihydroxypropyl dodecanoate observed for various formulations. The emulsion based 2,3 dihydroxypropyl dodecanoate resulted in rapid and complete or near-complete dissolution in both acid and neutral pH conditions, with or without added surfactant. In this embodiment, it is surmised that the effect observed in pigs is related to the enhanced biodistribution of 2,3 dihydroxypropyl dodecanoate in the gastric and intestinal lumen, such effect being due to the intact 2,3 dihydroxypropyl dodecanoate or metabolites or other related degradants thereof.

### Case Studies

Case study 1: Six subjects who had tested positive for COVID 19 by PCR and had current mild to moderate symptoms were instructed to take the 15 ml of the Liquid emulsion product (T1) 3 times daily with food. Ideally diluted in a glass of water 50 ml to 150 ml. The subjects were not screened for any inclusion criteria, other than not requiring hospitalisation and having 2 out of the 3 symptoms fever, sore throat and cough or shortness of breath. No placebo arm or other dosing control was considered for the study. Data collected was qualitative with some objective aspects which translated to symptom scoring as follows,

Fever Score
a. 0<37.8° C.,
b. 1 Reports mild sweats, chills or fatigue and has temperature of 37.8° C. to 38.5° C.
c. 2 Reports moderate discomfort sweats, chills or fatigue and temperature greater than 38.5° C. to 39.0° C.
d. 3 Reports requirement to be in bed and temperature greater than 39.0° C.

Sore Throat Score
a. 0 No discomfort
b. 1 Reports mild discomfort, able to speak and eat and drink
c. 2 Reports moderate discomfort requiring OTC medications for relief, pain eating drinking and speaking
d. 3 Reports severe discomfort, OTC medications providing some relief, pain causing reduced ability to take fluid and temporary elimination of solid food

Cough and Shortness of Breath Score
a. 0 No cough or shortness of breath
b. 1 mild occasional coughing or intermittent shortness of breath
c. 2 Persistent dry cough and/or short of breath for prolonged periods (several hours), not necessarily lying in bed
d. 3 Persistent dry cough and/or very short of breath requiring lying in bed

RESULTS: All subjects reported a reduction in their reported symptoms within 24 or 48 hours. No subjects reported new symptoms or any increased discomfort. The five subjects who reported fever at the start all reported no fever after 24 hours. The results are shown in Table 17.

TABLE 17

| | Fever | Fever | sore throat | Sore throat | cough | cough |
|---|---|---|---|---|---|---|
| SUBJECT | start | 24 hr | start | 24 hr | start | 48 hr |
| M01 | 1 | 0 | 2 | 0 | 0 | 0 |
| M02 | 0 | 0 | 2 | 0 | 3 | 1 |
| M03 | 3 | 0 | 2 | 1 | 3 | 1 |
| F01 | 3 | 0 | 3 | 1 | 3 | 1 |
| F02 | 3 | 0 | 2 | 1 | 1 | 0 |
| F03 | 2 | 0 | 1 | 0 | 0 | 0 |

CONCLUSION: These data support the conclusion that a composition comprising 2,3 dihydroxypropyl dodecanoate and an emulsifier is effective in the treatment of infectious disease such as respiratory tract infections. The data supports that a method of treatment of infectious disease such as respiratory tract infections in an animal subject such as a human subject in need thereof, comprising administering to the subject the compound 2,3 dihydroxypropyl dodecanoate and an emulsifier is effective.

Case study 2: Two subjects were provided with the Liquid emulsion formulation (T1) as a potential general health aid and were advised that it may or may not help to prevent symptoms of colds and flus. Surprisingly, both reported an immediate and lasting impact on gut health. Both reported cessation of chronic Diarrhoea.

SUBJECT 1. Female in her sixties had been suffering chronic diarrhoea for 4 years after her Gall bladder was surgically removed. Her episodes were several times daily and her lifestyle was severely affected. She reported difficulty in performing activities of daily living such as shopping, using public transport, working and exercise, for example swimming.

She self-administered a dose of 15 ml of Liquid emulsion (T1) with food once daily. After 72 hours her bowel movements had become normal. For five weeks she reported daily normal movements and stool consistency.

SUBJECT 2. Female in her thirties had been suffering chronic diarrhoea for “at least one year”. There was no obvious trigger or cause. Episodes were daily and seldom had a normal stool consistency.

She self-administered a dose of 15 ml of Liquid emulsion (T1) with food once daily. After one week she reported her bowel movements had become normal. For five weeks she reported daily normal movements and stool consistency.

CONCLUSION: The data supports a method of treatment of gastrointestinal upset, for example chronic diarrhoea in an animal subject such as a human subject in need thereof, comprising administering to the subject a composition comprising 2,3 dihydroxypropyl dodecanoate and an emulsifier is effective.

The composition of the invention comprising 2,3 dihydroxypropyl dodecanoate and an emulsifier, for example in liquid form such as a liquid emulsion, may be an effective aid to improved digestive health in some individuals who suffer chronic diarrhoea.

The words “comprises/comprising” and the words “having/including” when used herein with reference to the present invention are used to specify the presence of stated features, integers, steps or components but do not preclude the presence or addition of one or more other features, integers, steps, components or groups thereof.

It is appreciated that certain features of the invention, which are, for clarity, described in the context of separate embodiments, may also be provided in combination in a single embodiment, within the scope of the appended claims. Conversely, various features of the invention which are, for brevity, described in the context of a single embodiment, may also be provided separately or in any suitable sub-combination, within the scope of the appended claims.

1. A solid form composition comprising:
(i) 2,3 dihydroxypropyl dodecanoate, and
(ii) an emulsifier.

2. The composition of claim 1 wherein the composition comprises of from about 5 wt. % to about 95 wt. % 2,3 dihydroxypropyl dodecanoate based on the total weight of the composition.

3. (canceled)

4. The composition of claim 1 wherein the composition comprises of from about 2 wt. % to about 95 wt. % emulsifier based on the total weight of the composition.

5. (canceled)

6. The composition of claim 1 wherein the emulsifier selected from the group consisting of Fatty acids, Glyceryl-lacto esters of fatty acids, salts of fatty acids, mono glycerides of fatty acids, Lactylated fatty acid esters of glycerol and propylene glycol, Lactylic esters of fatty acids, sucrose oligoesters, sorbitol, polysorbitan, Lecithin, hydroxylated Lecithin, and Sodium Lauryl Sulfate, or combinations thereof.

7. (canceled)

8. The composition of claim 1 wherein the composition comprises one or more emulsifiers selected from the group consisting of short chain monoglycerides, short chain fatty acids, glyceryl polyethyleneglycol ricinoleate, and sucroglycerides or combinations thereof.

9. The composition of claim 1 wherein the composition comprises one or more emulsifiers selected from the group consisting of those obtained from plant extracts, for example those extracted from Quillaia, Yucca or seaweed.

10. The composition of claim 1 wherein the 2,3 dihydroxypropyl dodecanoate is encapsulated in the emulsifier.

11. The composition of claim 1 wherein the emulsifier is encapsulated in the 2,3 dihydroxypropyl dodecanoate.

12. (canceled)

13. (canceled)

14. (canceled)

15. The composition of claim 1 wherein the composition is in the form of a solid nanoemulsion.

16. (canceled)

17. (canceled)

18. The composition of claim 1 wherein the composition is coated with a polysaccharide, for example maltodextrin or inulin.

19. (canceled)

20. The composition of claim 1 wherein a single oral dose of the composition at a dosage of 0.025 g/kg body weight causes blood plasma levels of 2,3 dihydroxypropyl dodecanoate to exceed 3.9 micrograms/ml peak concentration within 6 hours.

21. The composition of claim 1 wherein dosing of the composition at a dosage of 0.025 g/kg body weight every 12 hours causes extracellular fluid in the lungs to have a concentration of 2,3 dihydroxypropyl dodecanoate which exceeds 3.9 micrograms/ml peak after 92 hours.

22. The composition of claim 1 wherein the composition further comprises a further pharmaceutically active agent.

23. The composition of claim 1 wherein the composition is microstatic and/or the composition is microcidal.

24. (canceled)

25. The composition of claim 1 wherein the composition further comprises one or more nutraceuticals.

26. (canceled)

27. (canceled)

28. (canceled)

29. (canceled)

30. A nutraceutical composition comprising the composition of claim 1.

31. A food composition comprising the composition of claim 1.

32. (canceled)

33. The composition of claim 1 for use as an anti-microbial agent.

34. The composition of claim 1 for use in the treatment of respiratory tract infections in mammals.

35. (canceled)

36. (canceled)

37. (canceled)

38. (canceled)

39. (canceled)

40. (canceled)

41. The compound 2,3 dihydroxypropyl dodecanoate for use in the treatment of infectious disease such as respiratory tract infections.

42. A method of treatment of infectious disease such as respiratory tract infections in an animal subject such as a human subject in need thereof, comprising administering to the subject the compound 2,3 dihydroxypropyl dodecanoate.

43. A method of treatment of infectious disease such as respiratory tract infections in an animal subject such as a human subject in need thereof, comprising administering to the subject the composition of claim 1.

44. A method of treatment of gastrointestinal upset, for example chronic diarrhoea in an animal subject such as a human subject in need thereof, comprising administering to the subject the composition of claim 1.

45. A method of modulating gastrointestinal microbiota in an animal subject such as a human subject in need thereof, comprising administering to the subject the composition of claim 1.

46. A method of modulating triglyceride metabolism, for example high density lipoprotein and/or low-density lipoprotein metabolism, in an animal subject such as a human subject in need thereof, comprising administering to the subject the composition of claim 1.

47. A method of reducing bilirubin levels in an animal subject such as a human subject in need thereof, comprising administering to the subject the composition of claim 1.

48. A method of increasing alkaline phosphate levels in an animal subject such as a human subject in need thereof, comprising administering to the subject the composition of claim 1.

49. A method of decreasing urea levels in an animal subject such as a human subject in need thereof, comprising administering to the subject the composition of claim 1.

50. (canceled)
